(19) United States
(12) Patent Application Publication
(10) Pub. No.: US 2010/0331254 A1
(43) Pub. Date: Dec. 30, 2010

(54) PULVERIZED FIBRIN CLOTS AND PHARMACEUTICAL COMPOSITIONS CONTAINING THEM

(75) Inventors: Ascher Shmulewitz, Tel Aviv (IL); Mazal Dahan, Mazkeret Batia (IL); Raphael Gorodetsky, Jerusalem (IL)

Correspondence Address: THE NATH LAW GROUP, 112 South West Street, Alexandria, VA, 22314, US

(73) Assignee: METAMOREFIX, Yarkona (IL)

(21) Appl. No.: 12/810,516

(22) PCT Filed: Dec. 25, 2008
(86) PCT No.: PCT/IL08/01676

§ 371 Date: Aug. 30, 2010

Related U.S. Application Data
(60) Provisional application No. 61/006,139, filed on Dec. 26, 2007.

(51) Int. Cl. A61K 38/36; C07K 14/745; A61P 43/00; A61P 17/00; A61P 19/04

(52) U.S. Cl. 514/13.6; 530/382; 428/402

(57) ABSTRACT

Provided is a pulverized fibrin clot and a pharmaceutical composition including a pulverized fibrin clot. The pharmaceutical composition may contain the pulverized fibrin clot suspended in a gel such as cross-linked hyaluronic acid. The pharmaceutical composition may be in a form suitable for injection and may be used, for example, in the treatment of connective tissue, such as skin connective tissue. Also provided is a method for preparing a pulverized fibrin clot as well as a method for treating connective tissue using the pharmaceutical composition.

### FIELD OF THE INVENTION

This invention relates to methods and systems for connective tissue treatment.

### BACKGROUND OF THE INVENTION

The following prior art publications are considered to be relevant for an understanding of the invention:
¹Radiat. Res. (1991) 125,181-186
²Journal of Investigative Dermatology (1999) 112, 866-872
³J. Lab. Clin. Med. (1997), in press
⁴Amer. J. Pathol. (1993),142,273-283
⁵J. Invest. Dermatol. (1982), 79,624-629
⁶Lab. Invest. (1986), 54,62-69
⁷J. Clin. Invest. (1985), 75,11-18
⁸J. Histochem. Cytochem. (1991), 39, 413-423
⁹NY Acad. Sci. (1986), 408, 228-235
¹⁰J. Lab. Clin. Med. (1994),124,339-347
¹¹Matsunuma, H., Kagami, H., Narit,a Y., Hata, K., On, o Y., Ohshima, S., and Ueda, M. Constructing a tissue-engineered ureter using a decellularized matrix with cultured uroepithelial cells and bone marrow-derived mononuclear cells. Tissue Eng 12, 509, 2006.
¹²Ramrattan, N. N., Heijkants, R. G., van Tienen, T. G., Schouten, A. J., Veth, R. P., and Buma, P. Assessment of tissue ingrowth rates in polyurethane scaffolds for tissue engineering. Tissue Eng 11, 1212, 2005.
¹³Hong, Y., Gao, C., Xie, Y., Gong, Y., and Shen, J. Collagen-coated polylactide microspheres as chondrocyte microcarriers. Biomaterials 26, 6305 2005.
¹⁴Taguchi, T., Xu, L., Kobayashi, H., Taniguchi, A., Kataoka, K., and Tanaka, J. Encapsulation of chondrocytes in injectable alkali-treated collagen gels prepared using poly(ethylene glycol)-based 4-armed star polymer. Biomaterials 26, 1247, 2005.
¹⁵Patent EP1490477 redifferentiated cells for repairing cartilage defects, French Margaret Athanasiou Kyriacos.

The human skin is the largest organ of the body, accounting for about 16% of the body's weight. It performs many vital roles as both a barrier and a regulating factor between the outside world and the controlled environment within the body.

There are two main layers of skin. The epidermis is made up of keratinocytes, which are stacked on top of each other. The keratinocytes develop at the bottom of the epidermis and rise to the surface, where they are shed as dead, hard, flattened cells. This layer is thus constantly being renewed. Melanocytes and Langerhans cells are other important cells of the epidermis.

The dermis consists mostly of connective tissue and is much thicker than the epidermis. It is responsible for the skin's pliability and mechanical resistance and is also involved in the regulation of body temperature. The dermis supplies the avascular epidermis with nutrients and contains sense organs for touch, pressure, pain and temperature (Meissner's corpuscles, Pacinian corpuscles, free nerve endings), as well as blood vessels, nerve fibers, sebaceous and sweat glands and hair follicles.

The subcutaneous layer is the fatty layer underneath the skin and consists of loose connective tissue and much fat. It acts as a protective cushion, insulates the body by monitoring heat gain and heat loss, and has a strong impact on the way the skin looks.

There are two distinct types of skin aging. Intrinsic aging is genetic in origin, while extrinsic aging is caused by environmental factors, such as exposure to sunlight. Intrinsic aging, also known as the natural aging process, is a continuous process that normally begins in the mid-20s. A number of extrinsic factors often act together with the normal aging process to cause premature aging of the skin. Most premature aging is caused by sun exposure. Other external factors that prematurely age the skin are repetitive facial expressions, gravity, sleeping positions, and smoking.

As the skin ages, the production of cells in the skin slows down and the cells become abnormally shaped, which adversely affects the texture of the skin:
Younger skin has more fat cells in the dermis than older skin. Thus, older skin looks more transparent and thinner than younger skin.
Certain components of the skin become depleted with age. The water-retaining and texture-enhancing elements in the intercellular structure such as ceramides, hyaluronic acids, polysaccharides, glycerin, and many others are exhausted and not replenished. Older skin thus tends to be drier than younger skin.
The skin's support structures, collagen and elastin, deteriorate or are damaged. Wrinkles form in damaged areas of the skin due to the decrease in elastin, collagen, hylauronic acid and other moisturizing reagents.
Older skin is more subject to allergic reactions, sensitivities, and irritation than younger skin due to a weakened immune system.
Dead skin cells do not shed as quickly and the turnover of new skin cells may decrease slightly.
For some unknown reason, the skin continues to grow and expand while the supporting fat tissues of the lower layers of skin and the bones recede.
Simultaneously, the facial muscles lose their shape and firmness. The skin thus begins to sag giving the face a drooping appearance.

It is very common to relate to wrinkles as damaged or wounded areas, thus relating to the corrective action as “wound healing”.

A substantial effort and large investment has been made worldwide aiming at fighting skin aging. Percutaneous application of collagen, vitamins and moisturizing and firming compounds are available. This requires at least daily application of these substances due to their very short half time life in the body.

Another approach is subcutaneous injections of dermal fillers. Permanent fillers are based mainly on silicone derivatives or a collagen matrix with non-biodegradible (poly-methylmethacrylate) spheres. The side effects of dermal filling include fibrosis, teratomas and facial distortions due to dislocation of the filler.

Temporary fillers are based on injections of biodegradable compounds such as collagen, synthetic polymers (cross-linked polyacrylamide, usually classified as hydrogels due to their water swelling and retaining properties), and various modifications of crosslinked and stabilized hyaluronic acid. These dermal fillers are injected subcutaneously about every 3-8 months.

Autologus fat implementation has also been used, but this involves a slow and painful healing process.

Fibrin clots are formed in vivo upon the reaction of fibrinogen and thrombin in the presence of calcium ions. The initial phase of wound healing starts after the formation of a fibrin clot, and involves the mobilization of cells from surrounding undamaged tissue. Normally, the earliest cells mobilized in the wound are inflammatory where they are active for a period of at least 1-3 days following injury. Subsequently, they are displaced by cells of the mesenchyme lineage which are immobilized in, navigate through, and digest, fibrin and replace fibrin with extracellular matrix (ECM) consisting of different collagen types, fibronectin and hyaloron. Endothelial cells also infiltrate the fibrin and generate microcapillary structures. Ultimately, these cells of the mesenchyme lineage replace the provisional fibrin matrix with granulation tissue populated by parenchymal cells and vasculature in secreted ECM.

Human fibroblasts are the major cellular entities responsible for the regeneration of the extracellular matrix (ECM) within the wound bed. Human fibroblasts also express specific membrane receptors to fibrinogen and thrombin. In the case of skin damage, human fibroblasts reform the matrix of the dermis. For example, during the course of healing of an incisional skin wound, human fibroblasts are mobilized from the surrounding tissue and enter into the fibrin clot, help dissolve it and generate as well as reform the collagens (i.e. type I and type III collagen) in the extracellular matrix. Based upon these properties of human fibroblasts, fibroblast implants have been suggested as a means for supplementing the body's natural wound healing regime^{1, 2}.

Purified “fibrin(ogen)” (a mixture of fibrin and fibrinogen) and several of its fragments (i.e. FPA, FPB, D and E) have been shown to be chemotactic to a variety of cells including macrophages, human fibroblasts (HF) and endothelial cells^{3,4,5,6,7}. Thrombin also has been shown to exert a proliferative effect on various cells including fibroblasts, endothelial cells, and to enhance wound healing in rats^{8, 9, 10}.

Recent tissue engineering techniques involve combining cells having regenerative potential, such as stem cells, either from embryonic sources or as freshly isolated cells, with an appropriate scaffold^{11, 12}. This technology allows engraftment and implantation of constructs with cells loaded onto the scaffold into tissue defects in an attempt to regenerate the damaged tissue. The current notion is to use a 3D biocompatible scaffold cell support, with adequate porosity to allow cells to enter into it and to allow exchange of nutrients and gases through the pore network. The cells are expected to proliferate and differentiate on the matrix^{13, 14}. Nevertheless, having to set up a tissue corrective procedure based on cell injection poses a huge barrier due to two major aspects: regulation and safety, as well as costs.

For example, recent research has shown that fibroblasts grown on a cartilage-like ECM environment can trans differentiate into normal chondrocytes, thus allowing repair of damaged cartilage tissue (also demonstrated to occur in vivo)¹⁵.

### SUMMARY OF THE INVENTION

In its first aspect, the present invention provides fibrin capable of binding to the surface of human cells such as fibroblasts and endothelial cells. The fibrin of the invention is in a pulverized form that may be prepared, for example, by milling or grinding dry and hardened fibrin clots. Thus, in its second aspect, the invention provides a method for preparing fibrin clots suitable for pulverization. In a preferred embodiment, the fibrin is clotted in the presence of a negatively charged polymer, (such as hyaluronic acid, one of its salts, or sodium alginate). Most preferably, the polymer is hyaluronic acid. The clot formed is heat dried into hardened brittle lump suitable for milling or grinding. Since the fibrin structures of the invention are based on a human protein, they are usually non inflammatory and nontoxic.

In its third aspect, the present invention provides a pharmaceutical composition for the treatment of damaged connective tissue, such as skin connective tissue or cartilage. The pharmaceutical composition of the invention comprises the pulverized fibrin of the invention. In a preferred embodiment of this aspect of the invention, the pharmaceutical composition contains the pulverized fibrin suspended in a gel matrix to form a stable suspension. In a preferred embodiment, the gel matrix is based on hyaluronic acid or one of its salts. The pharmaceutical composition is preferably in a form suitable for injection, and more preferably, in a form suitable for subcutaneous injection. The pharmaceutical composition of the invention tends to promote rejuvenation by binding and sequestering cells including stem cells, migrating through the skin tissue. The pulverized fibrin, being insoluble in the tissue environment, tends to immobilize cells in the skin tissue. The immobilized cells may secrete substances such as collagen, elastin, and hyaluronic acid which tend to accumulate in the skin and restore skin elasticity and smoothness. The pharmaceutical composition of the invention may promote regeneration of tissues. The composition of the present invention tends to attract endogenous fibroblasts into the damaged area, as opposed to the prior art which teaches implanting fibroblasts into connective tissue. The pharmaceutical composition may also include an analgesic such as lidocain.

In one embodiment, the fibrin structures are prepared by first preparing an aqueous solution comprising fibrinogen and an aqueous solution comprising thrombin and factor XIII. One or both of these solutions may contain an anionic polymer at a concentration of about 3-20 mg/ml. The anionic polymer may be, for example, a hyaluronic acid polymer or one of its derivatives, an alginic acid polymer or derivatives thereof, a cellulosic polymer or derivatives thereof (including carboxy methyl cellulose, Hydroxy propyl methyl cellulose, hydroxylpropyl cellulose, hydroxylethyl cellulose). The molecular weight of the anionic polymer is preferably about 0.5-5 million Daltons. The two solutions are combined to yield a final solution in which the ratio of fibrinogen:thrombin:factor XIII is preferably 5-100 mg/mL:1-100 U/mL: 1-50 U/mL, and most preferably 20-100 mg/mL:5-10 U/mL:2-20 U/mL. The clot formed is further dried and ground to form a powder that may then be suspended in an aqueous matrix based on a solution of a carrier gel.

In its fourth aspect, the present invention provides a method for treating connective tissue comprising injecting the pharmaceutical composition of the invention into the connective tissue to be treated. This aspect of the invention may be used, for example, for the treatment of skin connective tissue, or cartilage.

In a preferred embodiment, the gel matrix is based on an injectible polymer, capable of forming a gel-like texture or a high viscosity solution, such as a hyaluronic acid polymer or one of its derivatives, an alginic acid polymer or derivatives, a cellulosic polymer or derivatives (including carboxy methyl cellulose, hydroxy propyl methyl cellulose, hydroxylpropyl cellulose, hydroxylethyl cellulose), polyacrylamides, PLA(poly lactic acid) and PLGA (copoly lactic acid/glycolic acid). In a preferred embodiment the gel matrix is based on a naturally occurring polymer, existing in the human body, such as a polymer based on hyaluronic acid. Hyaluronic acid occurs either in a dissolved form as in the vitreous humor, synovial fluid and some tumor fluids, or as a gel as in the umbilical cord, in certain mesodermal tumors and in the dermis. The half life of hyaluronic acid in the tissue may be extended, for example, by chemical cross linking. Methods for hyaluronic acid (HA) cross linking are well known in the art. The hyaluronic acid can be cross linked through each of the 3 functional groups attached to its backbone:
Each repeating unit of hyaluronic acid contains one carboxylate group. These carboxylate groups can react with dihydrazides, such as adipic acid dihydrazide, succinic acid dihydrazide, with or without catalysis of EDC and/or sulfo-NHS (complete or partial cross linking).
Each repeating unit of hyaluronic acid contains four hydroxyl groups. These hydroxyl groups can react with di-epoxides, such as 1,4 butanediol diglycidyl ether, poly ethylene glycol diglycidyl ether and poly propylene glycol diglycidyl ether.
The hydroxyl groups can also react with dialdehydes to form acetal/hemiacetal derivatives under acidic conditions—a reaction that will lead to an ether cross linker.
Each repeating group of hyaluronic acid contains one acetamido group, which can go through deacetylization, leaving free amino groups. Amino groups can then cross link via formation of amides, imino or secondary amines.
The carboxylic group of the hyaluronic acid can react with a water soluble carbodimide to form O-acylisourea, which then will react with neighboring carboxyl to form an anhydride, which then will react with an hydroxyl group to give both inter- and intra-molecular crosslinks.

The pharmaceutical composition of the invention may be composed of various combinations of a cross linked and non cross linked hyaluronic acid polymers.

The fibrin structures of the invention may be used as a fibroblast binding scaffold for the repair of damaged cartilage, allowing migrating fibroblasts to be sequestered in the damaged cartilage area. The sequestered fibroblasts may eventually differentiate into chondrocytes.

The pharmaceutical composition of the invention may also be used as a lubricant in body joints, and may provide relief from pain caused by damaged or insufficient articular cartilage

Thus, in its first aspect, the invention provides a pulverized fibrin clot.

In its second aspect, the invention provides a pharmaceutical composition comprising a pulverized fibrin clot according to any one of the previous claims.

In another aspect, the invention provides a method for treating connective tissue comprising administering to an individual in need of such treatment a pharmaceutical composition comprising a pulverized fibrin clot.

In still another of its aspects, the invention provides a fibrin clot in a form suitable for pulverization.

In yet another of its aspects, the invention provides a method for preparing a fibrin clot in a form suitable for pulverization comprising:

(a) preparing a fibrin clot; and

(b) drying the fibrin clot.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows cell adhesion and proliferation of HFF to various matrices produced from purified fibrin(Nabi);

FIG. 2 shows the effect of heating of the fibrin particles on cell adhesion and proliferation to matrices (prepared from cryoprecipitate);

FIG. 3 shows the effect of thrombin on cell adhesion and proliferation to matrices (prepared from cryoprecipitate);

FIG. 4A shows wound healing formation in a 6 mm round sample of human facial skin implanted onto a CAM; and FIG. 4B shows the samples of FIG. 4A with an indication of the wound boundary;

FIG. 5 shows a graph of wound healing;

FIG. 6 shows histological staining of wounds by H&E (top row), Masson's trichrome (middle row) and Accustain staining (bottom row) of fibrin powder/HA (left column), HA (center column) and PBS (right column); and

FIG. 7 shows histological staining by Accustain (left column) and Masson's trichrome (right column) of non-implanted facial skin (top row), implanted and untreated skin (middle row) and treated implanted skin (bottom row).

### EXAMPLES

### Materials

### Fibrinogen and Thrombin

A mixture of fibrinogen and factor XIII, was obtained either from Nabi or as a cryoprecipitate of whole human blood. The Nabi mixture was Kohn fractionated fibrinogen, and was further purified by another Kohn fractionation cycle to produce a minimum of 75% clottable protein. Thrombin was obtained from Sigma. The activity of the thrombin was determined by clot time assays calibrated against an international standard (Vitex Inc. New York, N.Y.).

### Methods

Fibrin powders were prepared from a fibrinogen-factor XIII mixture, as follows: Fibrinogen was dissolved in Tris saline (pH 7.4), Tween 80 (2%), 5 mM NaCl and 1 mM CaCl₂, to a concentration of 20-60 mg/ml. Thrombin was dissolved in Tris saline (pH 7.4) to a 200 U/ml stock solution, added to a final concentration of 5-10 U/ml in the clotting solution. Polymer, such as Na—HA or Na-alginate was dissolved in phosphate buffered saline (PBS) to a concentration of 3-20 mg/ml (depending on the molecular weight, which ranged from 0.5-5 million Daltons). The clotting reaction was initiated by combining the fibrinogen/factor XIII solution and the thrombin solution with vigorous stirring.

In some experiments the fibrin powders were also prepared from fibrin glue/sealant kits, following the manufacturer's instructions for reconstitution.

In some experiments a polymer solution was added to one or both of the protein solutions. The clot formed in these reactions was either heated to 60-80° C. in a closed test tube for few hours and then air dried or dried at in an elevated temperature under vacuum to produce a hardened brittle clot. The hardened clot was then ground into particles of various sizes. The milling process was performed using a mortar and pestle which produced a powder with particles ranging in size between 20-250 microns. The smaller particles are suitable for injection using a small gauge needle, as would be required, for example, in the treatment of facial wrinkles, whereas the larger particles are suitable for procedures where injection with a larger gauge needle is possible, for example, when injecting into cartilage.

Adhesion of cells to the fibrin powder and proliferation of adhered cells on the fibrin powder was performed using the following assay. The fibrin powder was washed twice with saline then with HFF (Human foreskin fibroblasts) growth medium (DMEM high glucose medium supplemented with 10% fetal bovine serum, 2 mM L-glutamine, 100 U/ml Penicillin, 100 μg/ml streptomycin, 1% non-essential amino acids, 1.5 gr/1 Na2HCO3). Washing was performed by shaking the suspension. The powder was allowed to sediment and the medium was then removed by aspiration. The washings were performed to remove any residual ethanol (used for disinfection). The powder was then suspended in HFF growth medium at a final concentration of 5 mg/ml. Exponentially growing cultures of HFF, more than 50% confluent, were rinsed and detached from the substrate with Trypsin/EDTA. Trypsin activity was stopped by dilution by the addition of HFF Growth Medium. Cell concentration was adjusted to 22*10⁴ cells/ml. 1 ml of this cell suspension was added to 1 ml of the fibrin powder suspension. The tubes were closed loosely and covered with aluminum foil (to avoid any UV instability of the powder). The tubes were incubated at 37±1° C., 5±0.5% CO₂ and 95±5% relative humidity with gentle shaking. At the times indicated in the figures, the number of cells adhering to the fibrin powder was determined by removing unadhered cells from the suspension. The number of adhered cells was then determined using the MTT assay using a calibration curve and following the manufacturer's instructions.

In second set of experiments, the various preparations were observed for their wound healing effects, using the chorio allentoic membrane (CAM) system. This system utilizes the fact that human skin can be implanted on a CAM, where it vascularizes and survives for a few days (showing vitality and normal behavior), until it is rejected by the embryo. Human facial skin was removed during surgery, and maintained in saline, at 4° C. until use. A 6 mm disc was taken from a piece of skin. A “wound” was formed in the disc by punching a 2.5 mm hole in the disc to yield an annulus shaped implant that was implanted on a CAM within a few hours after surgery.

36 hours after implantation, a drop of a pharmaceutical composition of the invention was placed in the central hole of the implant. Additional doses of the pharmaceutical composition were administered as in Table 1. The implants were observed at a 1.5× magnification, at t=0, 2, 4, and 8 days after implantation. The wound was photographed and the contour of the wound was determined from the photograph by two independent observers. The area enclosed in each of the two contours was determined by Image J software, and the two areas were averaged together. The relative non-closure area after 8 days is defined by [Aₜ₌₈]/[Aₜ₌₀], where [Aₜ₌₀] is the wound area measured on day 0 (the day of implantation) and [Aₜ₌₈] is the wound area measured on day 8 (end of study). After photographing the wound for contour determination, the implants were separated from the CAM, and fixed for histological studies.

In a third study, fibrin structures were also produced by adding 10 U/ml of thrombin (Sigma) to a cryoprecipitate solution (purchased from the Israeli Blood Bank, fibrinogen concentration 25-30 mg/ml). The clots, some with hyaluronic acid (3-10 mg/ml) and some without, were heated to 65-75° C. or left at room temperature for 2 hours and then air dried overnight. The dried clots were than milled to a particle size of 20-250 μm. The powders were sterilized with 70% ethanol, and then re-dried. The various powders were suspended in a 10 mg/ml HA gel (carrier gel) at either 5 or 10 mg/ml. 40 μl of each of the suspensions were injected into a facial skin implant using a 27-30 G needle. The needle was slowly withdrawn while releasing the preparation so as to simulate injection along a wrinkle. The implants were sacrificed 6 days after injection and various histological stainings were performed.

TABLE 1
Preparation and dosing plan for test and control groups.

| | | | | | Application |
|---|---|---|---|---|---|
| Group | | Concentration | | Sterilization | Regime & |
| Mark | Content | (mg/ml) | Vicosity | Method | method |
| A | Hyaluronic | 10 | gel | Filter, 0.22μ | t = 0 |
| | acid/PBS | | | | t = 72 h |
| | | | | | syringe |
| B | Fibrin powder | 1. Fibrin particles-5 | Gel + | 1. Fib. Particles- | t = 0 |
| | suspended in | 2. HA-10 | (due to air | ETOH, 70% | t = 72 h |
| | HA/PBS | | entrapment) | 2. HA-filter, | pipette |
| | | | | 0.22μ | |
| Control 1 | PBS | — | solution | Sterile | t = 0 |
| | | | | | t = 48 h |
| | | | | | t = 96 h |
| | | | | | pipette |

### Histological Examination

All of the samples were fixed and preserved in formaldehyde. The samples were sliced and stained with haematoxylin and eosin to characterize changes in the epidermis. Two samples of each group were stained with Masson's trichrome (MG) to distinguish between collagen and myofibroblasts in order to determine the ratio of connective tissue to myofibroblasts in the wound area. Two samples were stained for elastin fibers (using Accustain kit).

### Results

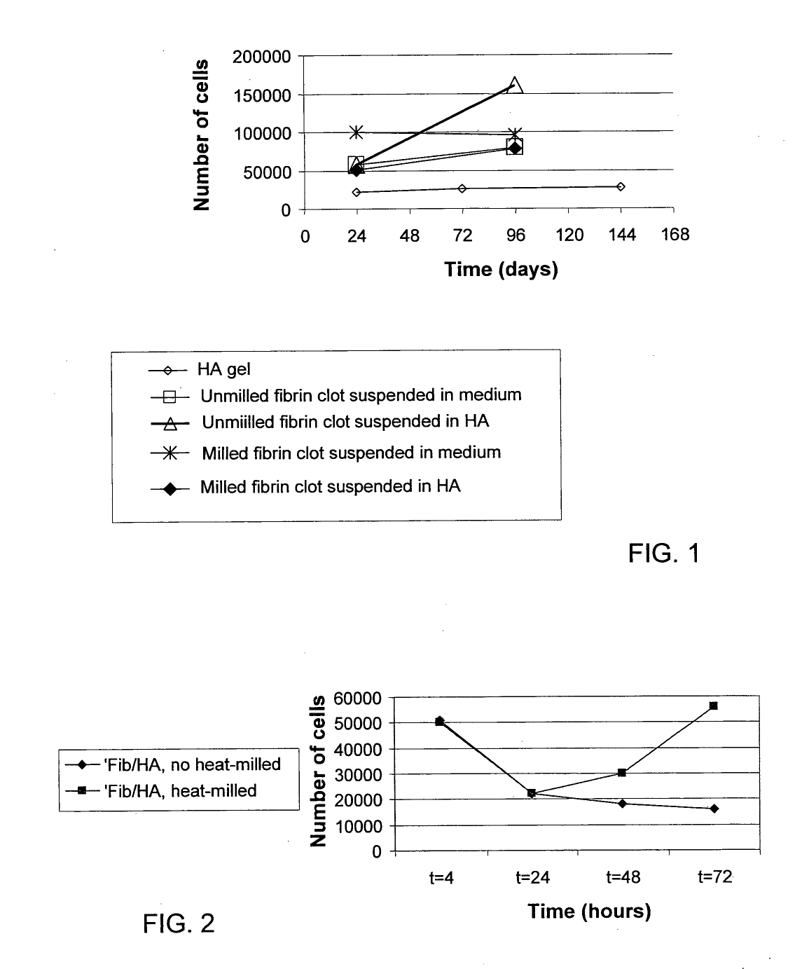

FIG. 1 shows adhesion of HFF to HA gel, unmilled fibrin clot suspended in medium, unmilled fibrin clot suspended in HA, milled fibrin clot suspended in medium, and milled fibrin clot suspended in HA. The fibrin clots used in FIG. 1 were prepared from the Nabi mixture. During the initial 24 hr incubation primarily cell adhesion, as opposed to cell proliferation, occurs. It was observed that the HA gel alone does not bind cells to a significant extent (the observed cells are probably free cells that did not precipitate due to the elevated viscosity of the HA gel). The unmilled fibrin clots suspended in medium, the milled clot in the presence of HA, and the unmilled clot in the presence of HA, induced about the same amount of cell adhesion. The milled fibrin clot suspended in medium showed the greatest cell adhesion, most likely due to the large exposed surface area and low viscosity. After the initial 24 hours, unadhered cells were removed, so that any increase in the number of adhered cells after that was due solely to cell proliferation. The unmilled fibrin clot in HA gel showed the highest rate of cell proliferation possibly due to the HA functioning as a nutrient. The unmilled fibrin clot in medium and the milled fibrin clot in the presence of HA showed about the same level of cell proliferation. The milled fibrin clot in medium showed no cell proliferation.

FIG. 2 shows HFF adhesion and proliferation to fibrin powder (milled fibrin clot) in HA prepared as above and fibrin powder in HA in which the heating step was omitted. The heat treated fibrin powder had a significantly enhanced proliferation capacity. This could be due to increased diffusion of HA into the clot during heating thus increasing the porosity of the fibrin clot or promotion of cell proliferation by the HA concentrated in the clot.

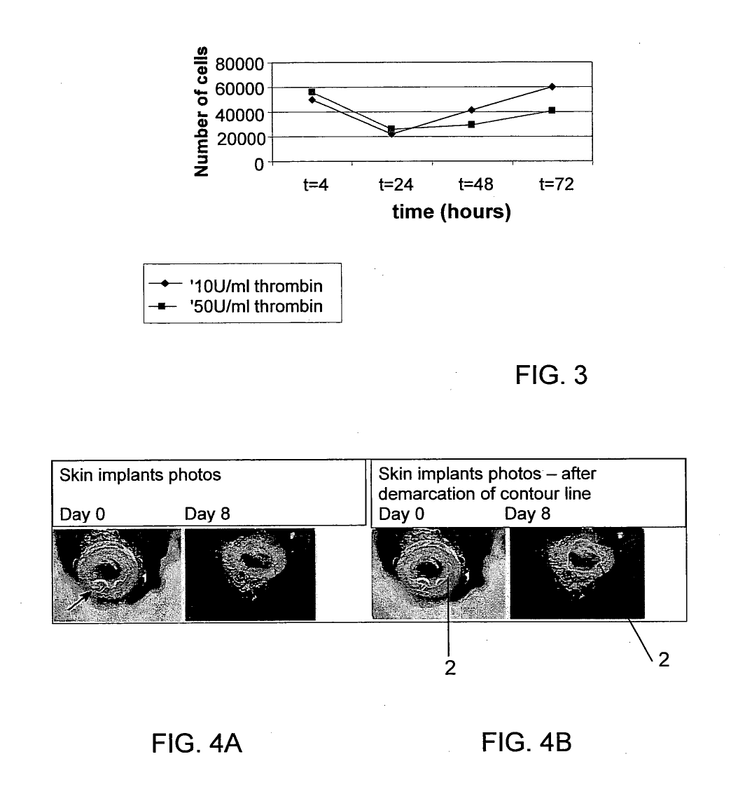

FIG. 3 shows HFF adhesion on milled heat dried clots in HA at two thrombin concentrations. Increasing the thrombin concentration increases the kinetic parameters of the fibrinogen scission, thus forming a more condensed (less porous) clot. FIG. 3 shows that with lower porosity of the clot (higher thrombin concentration) the rate of proliferation is decreased.

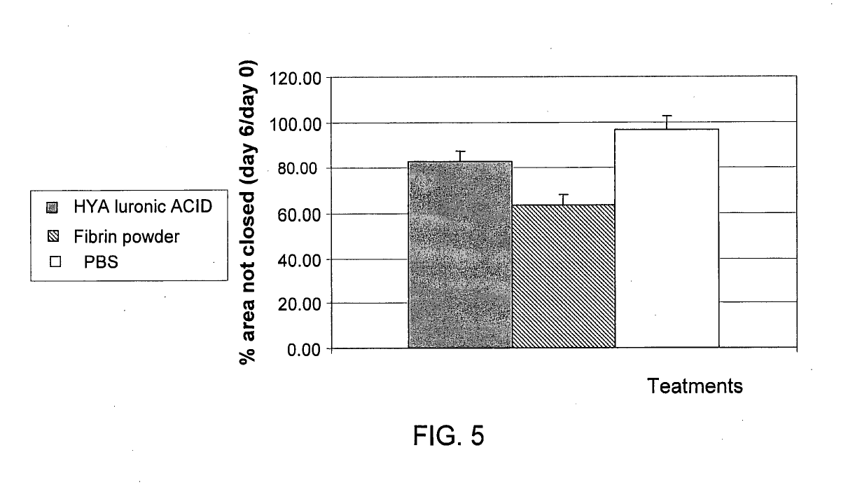

FIG. 4a shows an annular ring of human skin (indicated by arrow) implanted on a CAM on the day of implantation (left panel), and 8 days after implantation (right panel). FIG. 4b shows the photographs of FIG. 4a after superimposition of the contour line 2 of the wound. FIG. 5 shows the percentage of the original wound that had not healed after 6 days. The skin treated with the fibrin powder suspended in HA showed the best healing rate in comparison to treatment with HA alone or PBS alone.

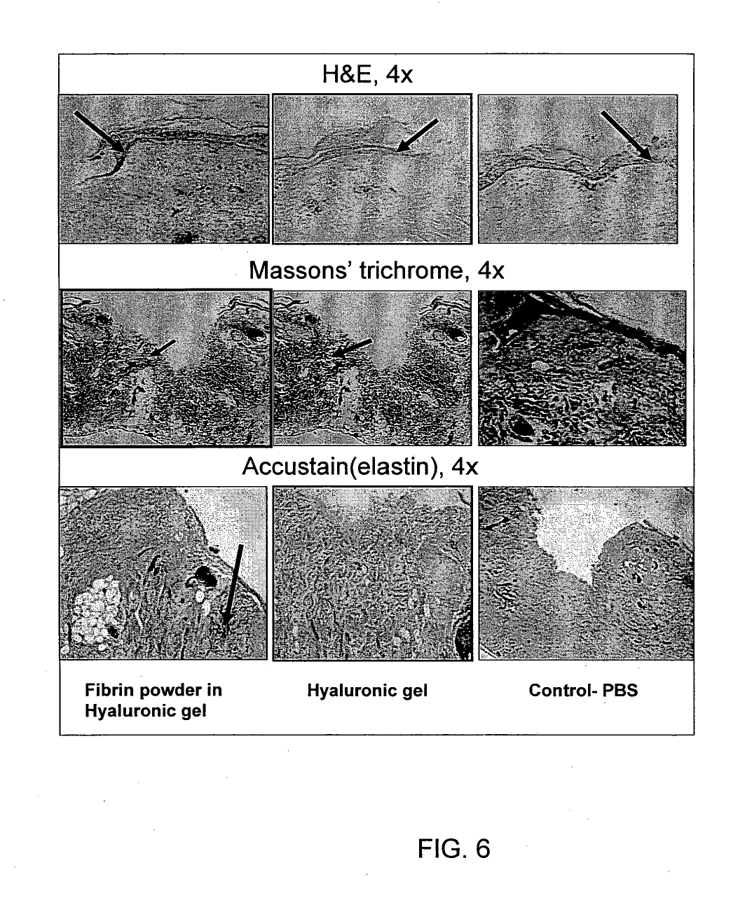

FIG. 6 shows histological staining of the wound edge of the implants (top row; H&E staining, middle row, Massons trichrome, bottom row accustain) on day 8 following topical administration of the various compositions. Thickening of the epidermis at the cut edge of the wound (indicated by arrow) is most pronounced in the fibrin powder treated implants and is an indication of epidermis closure, which is indicative of a healthy healing process. The Masson staining differentiates between collagen (which appears green) and muscles tissue (mainly myofibroblasts, appears brownish pink). The results show significantly more myofibroblasts in the wound of the fibrin (powder and HA) treated implants (indicated by arrow) in comparison to the controls. Blood vessels can also be detected and are stained brown. Their presence indicates a healthy healing process. The amount and thickness of the elastin fibers in the tissue (Bottom row, the elastin fibers appear black) determines the elasticity and tonus of the skin.

The results of FIG. 6 can be summarized as follows:
1. Fibrin powder/HA/PBS: The epidermis showed swelling and repair processes. The tissue in the wound area showed condensed muscle tissue and some collagen. In the Accustain staining, highly condensed elastin areas were found, mainly around the wound area. The fibers were very thick.
2. HA/PBS: The epidermis did not show significant healing, and appears very thin with no thickening. The tissue in the wound area contained very condensed muscle tissue and very little collagen. In the Accustain staining, some condensed elastin areas were found, mainly around the wound area. It was also significant that the fibers were thicker than those observed in the control groups.

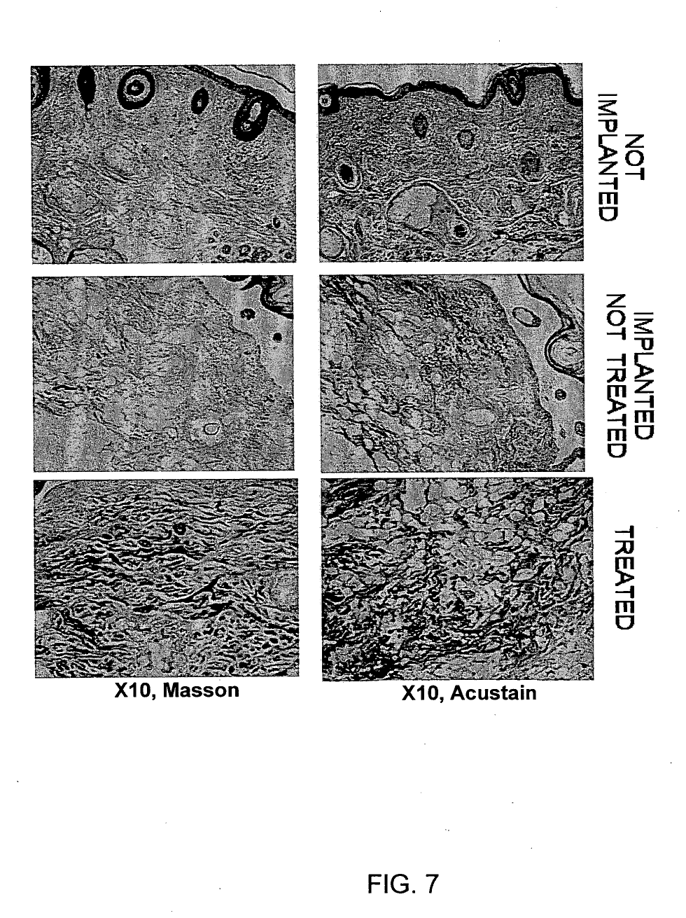

FIG. 7 shows Masson's trichrome staining and Accustain staining of three skin explants: a non-implanted skin explant (to define the baseline), an implanted but untreated skin explant (to define the effect of implantation on the skin) and an implanted skin explant treated by injection of a 10 mg/ml fibrin powder suspended in HA gel, where the fibrin powder was produced in the presence of HA and heated prior to a drying-milling stage.

The histological analysis shown in FIG. 7, clearly shows a dramatic increase in myofibroblasts (brown (dark) areas in Masson staining) in the connective tissue (green (light) stain in Masson), compared with the basic skin state (untreated). A semi-quantification process was used in order to evaluate the effect of the different preparations on the presence of fibroblasts in the dermis: the slices were observed microscopically (by two people) and evaluated using a qualitative scale of fibroblast presence: Whenever a high amount was observed the slice was rated as ‘++++’, whereas a slice devoid of myofibroblasts was rated as ‘− −’. Each rating was marked with a number to allow quantification of the observation (for a complete table of ratings vs. marks—see Table. 8). The results of the different processing parameters are shown in Table 9.

TABLE 8

| Observation | |
|---|---|
| Rating | Mark |
| −− | 0 |
| − | 1 |
| +−− | 2 |
| +− | 3 |
| + | 5 |
| ++ | 6 |
| +++ | 8 |
| ++++ | 10 |

TABLE 9

| TEST | | | Standard |
|---|---|---|---|
| GROUP | PROCESS PARAMETERS | Average Mark | Deviation |
| 1 | HA gel, 7 mg/ml | 3.2 | 1.8 |
| 2 | 5 mg/ml fibrin in 7 mg/ml HA gel. | 3.5 | 2.6 |
| | Preparation: 10 U/ml thrombin, | | |
| | Heating: 75° C. | | |
| 3 | PBS | 2.1 | 1.9 |
| 4 | 10 mg/ml fibrin in 7 mg/ml HA | 4.8 | 3.3 |
| | gel. | | |
| | Preparation: 10 U/ml thrombin, | | |
| | Heating: 75° C. | | |
| 5 | 10 mg/ml fibrin in 7 mg/ml HA | 3.6 | 2.4 |
| | gel. | | |
| | Preparation: 10 U/ml thrombin, | | |
| | Heating: Room Temperature | | |
| 6 | 5 mg/ml fibrin in 7 mg/ml HA gel. | 2.5 | 0.6 |
| | Preparation: 10 U/ml thrombin, | | |
| | Heating: Room Temperature | | |
| 7 | 5 mg/ml fibrin in 7 mg/ml HA gel. | 8.7 | 2.3 |
| | Preparation: 10 U/ml thrombin, in | | |
| | presence of 3 mg/ml HA Heating: | | |
| | Room Temperature | | |
| 8 | Non treated implants | 1.8 | 1.3 |
| 9 | Non implanted skin | 1.6 | 1.5 |

The non-implanted and the non treated skin indicate that the skin used in the study does not contain many fibroblasts, and is a very atropic dermis, indicative of aging skin. The PBS treatment does not seem to form any significant change in the skin, whereas an injection of Hyaluronic acid gel does seem to increase the concentration of fibroblasts in the dermis only slightly.
In general, whenever HA+fibrin powder was injected into the human dermal tissue, the presence of additional fibroblasts was observed compared with the other groups.
When semi quantifying the amount of fibroblasts in the tissue, one can notice a few trends, as follows:
   1. A potential dose response: Samples containing 10 mg/ml of fibrin powder (group 4) induce a higher amount of fibroblasts in the dermis, compared with 5 mg/ml of fibrin powder (group 2).
   2. The presence of Hyaluronic acid during clotting increases the amount of fibroblasts in the tissue dramatically (group 7).
   3. Heating (in the absence of HA) increases the presence of fibroblasts in the tissue (group 4 vs. group 5).

1.-23. (canceled)

24. A pulverized fibrin clot.

25. The pulverized fibrin clot according to claim 24, comprising particles having a size up to 500 micrometers.

26. The pulverized fibrin clot according to claim 25, comprising particles having a size up to 250 micrometers.

27. The pulverized fibrin clot according to claim 25, comprising particles having a size up to 100 micrometers.

28. The pulverized fibrin clot according to claim 24, comprising hyaluronic acid.

29. A pharmaceutical composition comprising the pulverized fibrin clot according to claim 24.

30. The pharmaceutical composition according to claim 29, further comprising an analgesic.

31. The pharmaceutical composition according to claim 29, wherein the pulverized fibrin clot particles are suspended in a gel.

32. The pharmaceutical composition according to claim 31, wherein the gel comprises hyaluronic acid.

33. The pharmaceutical composition according to claim 32, wherein the hyaluronic acid is cross-linked.

34. The pharmaceutical composition according to claim 32, wherein the hyaluronic acid is a mixture of cross-linked hyaluronic acid and unmodified hyaluronic acid.

35. The pharmaceutical composition according to claim 29, in a form suitable for injection.

36. The pharmaceutical composition according to claim 29, for use in treating connective tissue.

37. The pharmaceutical composition according to claim 35, for use in treating skin.

38. The pharmaceutical composition according to claim 36, for use as a dermal filler.

39. A method for treating connective tissue comprising administering to an individual in need of such treatment a therapeutically effective amount of a pharmaceutical composition comprising a pulverized fibrin clot.

40. A fibrin clot in a form suitable for pulverization.

41. A method for preparing a fibrin clot in a form suitable for pulverization, comprising:
(a) preparing a fibrin clot; and
(b) drying the fibrin clot.

42. The method according to claim 41, wherein the fibrin clot is dried by heating it to a temperature between 30° C. and 80° C.

43. The method according to claim 41, wherein the fibrin clot is air dried.

44. The method according to claim 41, wherein the fibrin clot is dried under vacuum.

45. The method according to claim 41, wherein the fibrin clot is prepared in the presence of a gel.

46. The method according to claim 45, wherein the gel comprises hyaluronic acid.
